(19) United States
(12) Patent Application Publication
(10) Pub. No.: US 2021/0350936 A1
(43) Pub. Date: Nov. 11, 2021

(54) FORMULA AND AXIAL LENGTH SPECIFIC IOL CONSTANT OPTIMIZATION

(71) Applicants: Carl Zeiss Meditec AG, Jena (DE); Carl Zeiss Meditec Inc., Dublin, CA (US)

(72) Inventors: Kyle Hunter Smith, Temple, TX (US); Jeremiah Robert Elliott, Temple, TX (US); John Cooper Bell, Temple, TX (US); Ian Deetz, Temple, TX (US); Craig Johnston, Temple, TX (US)

(21) Appl. No.: 17/241,724

(22) Filed: Apr. 27, 2021

Related U.S. Application Data
(60) Provisional application No. 63/020,092, filed on May 5, 2020.

(51) Int. Cl. G16H 50/70; A61F 2/16; A61B 34/10

(52) U.S. Cl. CPC G16H 50/70; A61F 2/16; A61B 2034/108; A61F 2240/002; A61B 34/10

(57) ABSTRACT

A method of determining a target IOL based on an optimized IOL constant, including, with a computing device, sorting available refraction data from a plurality of cataract surgery patients to identify appropriate post-operative refraction results based on user configurable criteria. Then, for each identified appropriate post-operative refraction result, the method determines a reference IOL constant that would have given an exact desired result for a selected IOL power estimation formula and analyzing the reference IOL constants to provide at least one optimized IOL constant that is: sub-grouped according to an axial length for the selected IOL power estimation formula; or determined as a function of axial length. The computing device determines based on the at least one optimized IOL constant, a target IOL power for a target axial length and target IOL power estimation formula; and provides, based on the target IOL power, a target IOL implant.

### RELATED APPLICATION

The present application claims the benefit of U.S. Provisional Application No. 63/020,092, filed May 5, 2020, entitled “Formula and Axial Length Specific IOL Constant Optimization,” which is hereby incorporated herein in its entirety by reference.

### TECHNICAL FIELD

Embodiments of the invention relate generally to intraocular lens (IOL) implants implanted in the eye related to cataract surgery. More specifically, embodiments of the invention relate to formulas and axial length specific intraocular lens constant optimization to facilitate the accurate determination and application of intraocular lens implants.

### BACKGROUND

Cataract surgery relates to the removal of the natural crystalline lens of the eye that has become cloudy, discolored or opaque due to age, disease, medication side effects or other factors. Various studies have demonstrated the value of personal and global intraocular lens implant constant optimization to improve the accuracy of IOL implant prescriptions that are placed in the eye during surgery. A variety of different intraocular lens implant calculation formulas are utilized to select the prescription for an intraocular lens that is to be placed in the eye.

Patients who undergo cataract extraction now have higher expectations as to visual outcome than they did in the past. In addition, many of these patients have previously had corneal refractive surgery for example, radial keratotomy or laser refractive surgical procedures. This complicates the task of determining the appropriate power for an intraocular lens implant because the cornea is altered from its natural shape in refractive surgical procedures. Further, patients who have previously undergone corneal refractive surgical procedures generally have a higher motivation to function visually without wanting to rely on eye glasses or contact lenses as visual aids. While this is not always possible, it is the goal of many patients who are about to undergo cataract extraction. In addition, cataract surgery patients generally have more knowledge and understanding of options available to them and have greater access to information regarding procedures and lens options than in previous years.

Much information that is related to patients involved in cataract surgery is stored in electronic medical records (EMR). There are many electronic medical record systems in which information is stored. There is little consistency in the systems and access to the information they contain may be limited.

A variety of IOL calculation formulas (generally referred to herein as “IOL power estimation formulas”) are utilized to calculate the appropriate power for an intraocular lens implant. Many formulas have been published and unpublished/proprietary IOL formulas exist as well. These formulas include but are not limited to the Holladay I, Hoffer II, SRK/T, Haigis, Holladay II, Olson and Barrett II formulas. These formulas are generally based on measurements of the corneal curvature or K measurement and axial length (AL) of the eye. Other measurement factors may be included as well. In addition to AL and K, many formulas include a single IOL constant. Some formulas include multiple IOL constants. Some of the formulas also take into account combinations of anterior chamber depth (abbreviated ACD), preoperative refractive measurements and/or preoperative thickness of the natural crystalline lens. The Holladay II formula, for example, is based on seven measurements, including the patient's age and the horizontal white to white (WTW) measurement. At least one formula uses a theoretical model eye in which ACD is related to AL and K, and is also determined by the relationship between the A-constant and a “lens factor.” The position of the principle plane of the IOL may also be considered to be a relevant variable. Many of these formulas are generally available and known to those skilled in the art.

Some intraocular lens implant formulas are mathematical formulas in a conventional sense that can be recognized as such by one skilled in the art. More often the formula is much more complex. Some formulas include thousands of lines of computer code. Some formulas are made available in the form of a DLL (dynamic link library file) which is a kind of computerized “black box” that conceals the working of the formula but permits a user to make input and receive output without being able to see the workings of the formula. These are however available to those skilled in the art often with the payment of royalties. However, access to these formulas provides those skilled in the art with the ability to make and use the inventions disclosed and claimed herein. Some formulas are particularly adapted for application in the context of eyes that have had corneal refractive surgery prior to undergoing cataract surgery. These include, for example, the Barrett True-K formula.

Generally, an eye surgeon has a constant that they have identified through experience or for other reasons that is a specific numerical value that they use that can be applied to each of the various intraocular lens calculation formulas. For example, a typical surgeon may know that the constant with which he or she has had success is 119.3. This constant is then used by the surgeon in a general manner with one of many formulas to calculate the power for an IOL.

One system for calculating IOL constants known in the prior art is the Alcon ORA system with AnalyzOR technology. This prior art system allows users to manually enter postoperative refraction data through a web portal. The system then performs IOL constant optimization calculations for individual surgeons (personal constants) and for the community of surgeons that use the proprietary ORA system (global constants).

The prior art system generates an optimized constant for each IOL model for each of 6 subgroups listed below.
1 no prior refractive surgery
2 post myopic LASIK with an axial length less than 26 mm
3 post myopic LASIK with an axial length greater than 26 mm
4 post hyperopic LASIK
5 post radial keratotomy with 4 cuts
6 post radial keratotomy with 8 cuts

These optimized constants are proprietary and specific for use with the ORA calculations for IOL spherical and toric lens power. Accordingly, there are at least two shortcomings of the prior art system.

First, the optimized constants are not applicable to various IOL power estimation formulas used by surgeons worldwide who do not use the proprietary system. The optimizations only apply to the proprietary formula that is intrinsic to the particular prior art system. Second, The known prior art does not subcategorize the optimized constants into axial length related groups except for post myopic LASIK eyes with long axial lengths. This may result in less than ideal refractive outcomes which may leave cataract surgery patients unsatisfied in comparison to their expectations.

Accordingly, there is still significant room for improvement in the calculation of intraocular lens implant power in the context of cataract surgery.

### SUMMARY

Various studies have demonstrated the value of personal and global IOL constants. Personal IOL constant determination relates to constants that apply specifically to a single surgeon based on a data set of prior outcomes from surgery performed by that surgeon. Global IOL constants relate to constants that are arrived at based on a data set including multiple surgeons either at a single surgical practice or at many surgical practices. Other systems in the prior art, such as the Alcon ORA System described herein, can generate personal and global IOL constants. However, a number of problems remain that are unsolved with these determinations.

For example, an IOL constant (or constants) determined for a single formula may not necessarily be appropriate for use with other IOL power estimation formulas. For example, separate IOL constants may be determined for Holladay I and Hoffer II formulas. This may be referred to as formula specific IOL constants.

In addition, IOL power estimation formulas that require a single IOL constant as one of the data inputs may not provide optimum results across the full spectrum of axial lengths. One common manifestation of this problem is that, for many years, surgeons have used certain estimation formulas for eyes of average axial length and other formulas for short or long eyes. They have done this because no single formula has proved to provide optimum results across all axial lengths for optimum outcomes. This may be referred to as Axial Length Specific IOL power estimation formulas.

The relative axial length of a patient's eye has been a contributing factor that has affected the reliability of IOL power estimation formulas. Unusually short or long eyes are more likely to have post-operative results that deviate significantly from the predicted spherical equivalent generated by the IOL power estimation formulas. Embodiments of the invention address many of the above problems by optimizing IOL constants to account for variations in axial length and are expected to improve refractive outcomes following cataract surgery and intraocular lens placement, thus providing patients with better postoperative vision and greater satisfaction. This is a benefit to surgeons as well.

Example embodiments of the invention utilize a large data set of cataract surgery cases to generate a series of optimized reference IOL constants as a function of axial length for one or more IOL power estimation formulas, for individual surgeons (personal constants), for a larger group of the community of surgeons (global constants). As discussed further below, some embodiments of the invention relate to optimization of formula specific IOL constants in conjunction with optimization based on more than one axial length. This ability to generate optimized IOL constants for available IOL power estimation formulas used by surgeons around the world can be used to provide more reliable and accurate data for use by the surgeon leading to improved surgical results.

In some embodiments, the invention includes the following:

A method of determining a target IOL based on an optimized IOL constant, comprising with a computing device:
sorting available refraction data from a plurality of cataract surgery patients to identify appropriate post-operative refraction results based on user configurable criteria;
for each identified appropriate post-operative refraction result, determining a reference IOL constant that would have given an exact desired result for a selected IOL power estimation formula;
analyzing the reference IOL constants to provide at least one optimized IOL constant that is:
   sub-grouped according to an axial length for the selected IOL power estimation formula; or
   determined as a function of axial length;
determining based on the at least one optimized IOL constant, a target IOL power for a target axial length and a target IOL power estimation formula; and
providing, based on the target IOL power, a target IOL implant.

The method may further include gathering the post-operative refraction data from pre-existing electronic medical records by computer-based analysis via the computer interface.

The method may further include performing separate computerized analysis and optimization for short, long and average axial length eyes.

The method may further include analyzing the reference IOL constants to provide at least one optimized IOL constant is sub-grouped according to an axial length for the selected IOL power estimation formula.

The method may further include analyzing the reference IOL constants to provide at least one optimized IOL constant further comprises, for each sub-group:
determining a mean IOL constant for the sub-group using the reference IOL constants;
excluding a set of outlier reference IOL constants that are at least two or more standard deviations from the mean IOL constant;
recalculating a second mean IOL constant from the remaining values that have not been excluded; and
reporting the recalculated mean IOL constant as the optimized IOL constant.

The method may further include outputting separate optimized IOL constants for at least short, long and average axial length sub-groups.

The method may further include analyzing the reference IOL constants to provide at least one optimized IOL constant is determined as a function of axial length.

The method may further include analyzing the reference IOL constants to provide at least one optimized IOL constant comprises preforming linear regression analysis on the reference IOL constants to provide an optimized IOL constant determined as a function of axial length:

The method may further include:
receiving a user selected target axial length; and
determining an optimized IOL constant using the user selected target axial length.

The method may further include selecting the target IOL power estimation formula from a group consisting of Barrett Universal II, Barrett True-K with refraction, Barrett True-K with no Refraction, Holladay I, Hoffer II, SRK/T, Haigis, Holladay II and Olson.

The method may further include ordering the target IOL.

The method may further include a non-transitory computer readable data storage medium that is not a carrier wave or signal comprising program code to carry out the method of any one of the previous claims 1-11.

The method may further include a computerized planning device configured to determine a target IOL based on an optimized IOL constant, comprising a display unit; a user input device; and processing circuitry, the processing circuitry configured to:
sort available refraction data from an EMR database comprising available refraction data from a plurality of cataract surgery patients to identify appropriate post-operative refraction results based on user configurable criteria;
determine a reference IOL constant for each identified appropriate post-operative refraction result that would have given an exact desired result for a selected IOL power estimation formula;
calculate, based on the reference IOL constants, at least one optimized IOL constant that is:
   sub-grouped according to an axial length for the selected IOL power estimation formula; or
   determined as a function of axial length, and
display the optimized IOL constant.

The computerized planning device may further include wherein the processing circuitry is configured to:
determine based on at least one optimized IOL constant, a target IOL power for a target axial length and target IOL power estimation formula; and
order based on the target IOL power a target IOL.

The computerized planning device may further include wherein the processing circuitry is configured to perform separate optimization for short, long and average axial length eyes, and provide at least three optimized IOL constants for each of the short, long and average axial lengths.

Embodiments of the invention further include a computerized method of formula and axial length specific IOL constant optimization, comprising:
electronically sorting available refraction data from a plurality of cataract surgery patients to identify appropriate post operative refraction data based on user configurable criteria;
identifying eligible surgical cases for optimization;
identifying a constant value for each surgical case that would have given an exact desired result with a selected IOL formula;
analyzing a series of the constant values to generate a mean constant value and to define the constant values that deviate by more than two standard deviations from the mean constant value;
excluding the constant values that deviate by more than two standard deviations from the mean;
recalculating a second mean constant value from remaining cases that have not been excluded; and
reporting the recalculated mean constant value as an optimized constant; and
using the optimized constant in at least one future IOL calculation;
using the result of the at least one future IOL calculation to select an IOL for implantation; and
implanting the IOL.

The method may further include gathering the post operative refraction data from pre-existing electronic medical records by computer-based analysis via a computer interface.

The method may further include performing separate computerized analysis and optimization for short, long and average axial length eyes.

The method may further include performing separate computerized analysis and optimization for at least two different IOL calculation formulas.

The method may further include outputting separate constants for at least short, long and average axial length eyes.

The method may further include performing separate computerized analysis and optimization to a continuous range of axial lengths through a non-linear regression analysis.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a flowchart depicting a method according to an example embodiment of the invention;

FIG. 2 is a block diagram of a computerized analysis system according to an example embodiment of the invention;

FIG. 3 is a depiction of a graphical user interface according to an example embodiment of the invention; and

FIG. 4 is a detailed view of the graphical user interface depicted in FIG. 3.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

Example embodiments of the invention correlate surgical cases that make up the data set used in calculating optimized IOL constants into groupings based on the axial length of the eye or as a function of axial length or create constants optimized to specific IOL estimation formulas. The invention then, for example, calculates an optimized IOL constant for each axial length group or for a target axial length and specific for each IOL power estimation formula. Even though some IOL power estimation formulas have been proposed and utilized to estimate IOL prescriptions for certain ranges of axial lengths, embodiments of the invention add variability to account for axial length specific adjustments to each specific IOL power estimation formula by creating an IOL constant specific for each axial length group or determined using axial length as an input. So far as is known to the applicant, no prior art device or application performs, generates and displays IOL constant optimizations specific for short, average and long eyes. Further, no prior art device or application known to Applicant performs, generates and displays IOL constant optimizations specific for each IOL estimation formula.

Example embodiments of the invention also enable users to manually enter post-operative refraction data for determining personal IOL optimization constants. Some embodiments of the invention display each IOL power estimation formula, and IOL constants either optimized for a plurality of different axial lengths or as a function of axial length so the user can select the constant(s) that are most appropriate for the surgeon's preferred IOL power estimation formula and for the target eye's axial length. Example embodiments of the invention for example, divide the surgical cases into at least three axial length groups: short, average, and long and provide optimized IOL constant tailored for each axial length group for each IOL power estimation formula. The principle, however, can be applied to any number of groupings thereby making the analysis more granular, or even to a continuous range of axial lengths through a regression analysis of the data as disclosed herein. The latter approach may be more practical for global constants because of a large patient data set may be needed, but may be applied to personal constants in some embodiments.

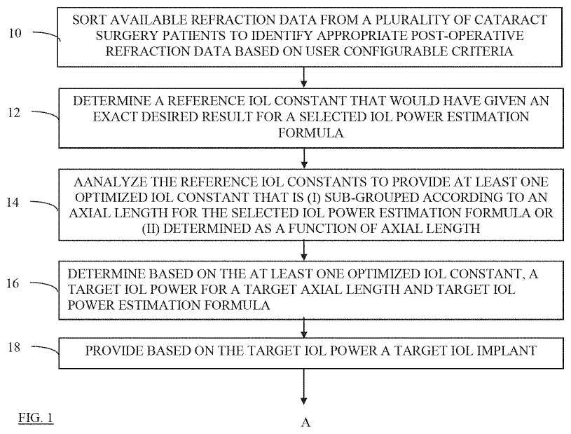

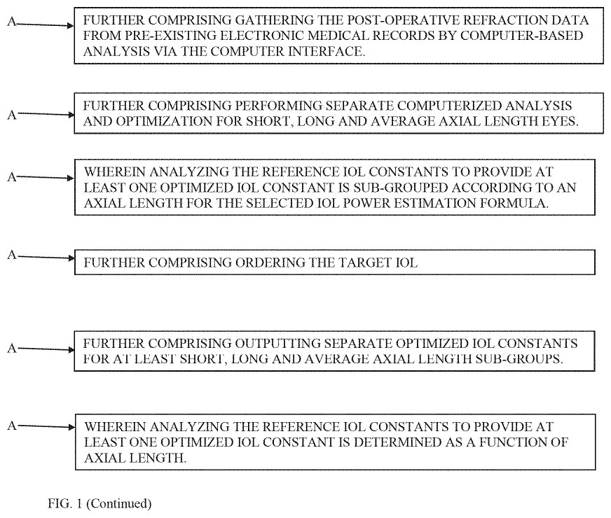

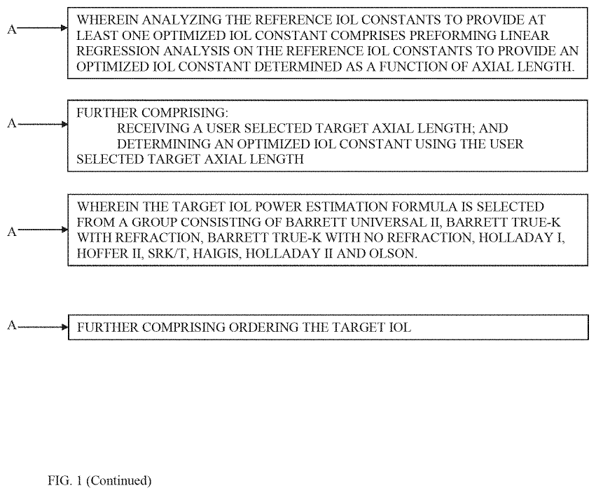

FIG. 1 is a flowchart depicting a method of formula and axial length specific IOL constant optimization in accordance with an example embodiment of the invention. The method includes electronically sorting available refraction data from a plurality of cataract surgery patients to identify appropriate post-operative refraction data based on user configurable criteria (10), determining a reference IOL constant that would have given an exact desired result for a selected IOL power estimation formula (12), analyzing the reference IOL constants to provide at least one optimized IOL constant that is (i) sub-grouped according to an axial length for the selected IOL power estimation formula or (ii) determined as a function of axial length (14), determining based on the at least one optimized IOL constant, a target IOL power for a target axial length and target IOL power estimation formula (16), and providing based on the target IOL power a target IOL implant (18).

The first step in the optimization process is to electronically sort available refraction data from a plurality of cataract surgery patients to identify appropriate post-operative refraction data based on user configurable criteria (10). In some examples, the available refraction data may be provided from post-operative refraction data from pre-existing electronic medical records (EMR) via a linked computer data base. For example, ZEISS VERACITY Surgical is a web-based software application designed to help cataract surgeons plan cataract surgery. It interfaces with various electronic medical record (EMR) systems to extract post-op refraction data. The disclosed process can use the post-op refraction data and the surgical planning data generated by VERACITY to generate the optimized reference IOL constants. With this EMR integration, embodiments of the invention are able to optimize reference IOL constants with no additional effort or manual data entry by the user. The post-op refractions are automatically imported from the EMR system. Other suitable EMR record information may also be used and may include web-based, manually entered, or other stored types of referenced data.

The reference data may be sorted and filtered based on patient history, including for example, prior surgical procedures performed on the patient's eye, the specific surgeon or practice group performing the referenced procedure (e.g., for establishing personalized constants or global constants); physical criteria of the referenced patient data including, for example, the referenced patient's age, the axial length of the referenced eye (e.g., short, average, or long eye lengths), ACD, other anatomical measurements of the eye, or the like.

Once sorted, eligible cases for optimization are identified that correspond with the user configured criteria. For example, cases pertaining to only those performed by a specific surgeon (e.g., for personal constants) or those relating to a specific surgical group may be selected for further evaluation.

Next, the process determines a reference IOL constant that would have given an exact desired result for a selected IOL power estimation formula (12). The mathematical process for optimization, using the eligible cases in the data set, is relatively straightforward and involves determining an IOL constant for each surgical case, for one or more IOL power estimation formulas that would have given the exact desired surgical result (e.g., the exact IOL implant). In other words, in a simple example case the IOL power estimation formula such as the Barrett Universal II is mathematically rearranged and solved to identify the reference IOL constant or constants if there is more than one that would have resulted in the optimal IOL implant power determination for the given data set. Additionally, or alternatively, statistical or integral analysis techniques may be utilized to determine the reference IOL constant. The optimal IOL constants may then be collected and either categorized based on axial length groups (e.g., short, average, or long) or otherwise correlated to axial length values for subsequent regression analysis as described herein.

The reference IOL constants may be calculated using the selected surgical cases for various IOL power estimation formulas. Such IOL power estimation formulas may include, but are not limited to, Barrett Universal II, Barrett True-K with refraction, Barrett True-K with no Refraction, Holladay I, Hoffer II, SRK/T, Haigis, Holladay II, Olson and the like. The techniques described herein also have the benefit of working with proprietary or “black box” type power estimation formulas where the particulars of a given formula are concealed from the user. The system may back calculate the optimal reference IOL constant by adjusting relevant input information for these formulas until a reference IOL constant provides the target IOL power recommendation. Additionally, or alternatively, the system may be applied to novel IOL power estimation formulas.

The method of FIG. 1 further includes analyzing the reference IOL constants to provide at least one optimized IOL constant that is (i) sub-grouped according to an axial length for the selected IOL power estimation formula or (ii) determined as a function of axial length (14). In some embodiments, this analysis may be performed by generating a mean constant value categorized by axial length, discarding any constant values that deviate by more than two standard deviations from the referenced mean, and recalculating the mean from remaining cases that have not been excluded, and reporting the recalculated mean value as the optimized IOL constant. For example, the reference IOL constants (e.g., those determined for the referenced data set) are grouped together depending on the relative axial length of the referenced eye (e.g., grouped according to short, average, or long axial lengths). The mean IOL constant value is then determined for each axial length group. These sub-grouped mean values are then further optimized by determining a set of outlier data corresponding to those referenced IOL constants that are at least two standard deviations away from the mean value and removed from the data set for the referenced group. The sub-grouped mean values are then recalculated based on the reduced data sets to provide a revised, optimized mean value IOL constant for each axial length group. These optimized IOL constants are then reported to the user for consideration and selection in accordance to a target IOL power estimation formula and target axial length.

Additionally, or alternatively, the optimized IOL constants may be calculated and provided as a function of axial length to provide a more tailorable IOL constant. For example, the reference IOL constants may be correlated with the axial length of the reference eye. Suitable regression analysis (e.g., linear regression analysis) may be performed on the data set to provide an optimized IOL constant as a function of axial length. For a given patient, the user (e.g., surgeon) may input a target axial length (e.g. the patient's axial length) and the disclosed process may return an optimized IOL constant for the target axial length using the regression analysis.

In some embodiments, the optimized IOL constant may be calculated and displayed for at least three different axial lengths (e.g., short, average, and long) for one or more IOL power estimation formulas. Additionally, or alternatively, the analysis and optimization may be performed for a continuous range of axial lengths through a regression analysis allowing the user to input a target axial length and receive an optimized IOL constant for one or more IOL power estimation formulas for that axial length.

The resultant optimized IOL constant is provided via a system display to the user for selection and subsequent use. Any additional data relevant to the optimized IOL constants may be displayed along with the constants. Such additional data may include, but are not limited to, the number of patient record data included in determining the optimized IOL constant, the number of patient record data available for calculation, the optimized global or personalized IOL constant as an alternative value, calculated IOL constants optimization for two or more different IOL calculation formulas, and the like.

The disclosed optimization process may be performed automatically, on a daily basis, or other selected basis on the user's system. Users may also configure the system to automatically update their constant selections to the latest optimized constants, or they can opt to update their selections manually. In some embodiments, the disclosed process may be part of a web-based application to allow for higher accessibility, scalability, or flexibility. Additionally, or alternatively, the disclosed process can also be deployed in a client server model. Automated importing of data from integrated EMR systems may also be used to potentially help reduce errors associated with manual data entry. Nevertheless, essential features of the invention can be made to function in a system that relies on manual data entry.

The method of FIG. 1, further includes using the optimized IOL constants in a future IOL calculations by determining, based on the at least one optimized IOL constant, a target IOL power for a target axial length and target IOL power estimation formula (16), and providing based on the target IOL power a target IOL implant (18). For example, the user may select a desired optimized IOL constant depending on the preferred IOL power estimation formula and target eye axial length that will be operated on. Based on the selected optimized IOL constant the system may perform the selected IOL power estimation formula to determine a target IOL power for the patient. An IOL implant having the target IOL power may then be provided to the surgeon and implanted in the patient's eye by an appropriate surgical procedure.

FIG. 2 is a block diagram of a computerized IOL constant optimization system 50 according to an example embodiment of the invention configured to perform one or more of the IOL constant optimization procedures disclosed herein. System 50 includes user display 52, input/output device(s) 54, and processing circuitry 56. System may be linked (e.g., wireless or wired connection) to EMR database 60 and surgical system 70. System 50 may be configured to analyze and provide one or more optimized IOL constants for one or more IOL power estimation formulas that are a function of axial length and/or based on user specified criteria. As disclosed above, the user may select or filter search criteria for EMR database 60 based on desired eye axial length, other patient identifiers, clinician information, IOL power estimation formulas, and the like. Once selected, processing circuitry 56 may determine IOL constants value for each identified surgical case that would have given an exact desired result with a selected IOL power estimation formula. The processing circuitry 56 may then generate an optimized mean value IOL constant based on the determined IOL constants and a plurality of axial length sub-groups or a regression analysis to provide an optimized IOL constant as a function of axial length. The optimized IOL constant may be displayed via display 52 to allow the user to use the IOL constant to perform a subsequent IOL power estimation and obtain a target IOL power and implant for the patient.

The disclosed techniques may be implemented, at least in part, in hardware, software, firmware, or any combination thereof. For example, various aspects of the described techniques may be implemented within one or more processors, including a microprocessor, application specific integrated circuit (ASIC), digital signal processor (DSP), field programmable gate array (FPGA), or appropriate integrated or discrete logic circuitry, as well as any combinations of such components. The term “processor” or “processing circuitry” may generally refer to any of the foregoing logic circuitry, alone or in combination with other logic circuitry, or any other equivalent circuitry. A computing device including hardware may also perform one or more of the techniques of this disclosure.

The disclosed firmware, hardware, software, and the like may be implemented within the same device or within separate devices to support the various techniques disclosed herein. In addition, any of the described components may be implemented together or separately as discrete but interoperable logic devices. The disclosed components of system 50 is intended to highlight different functional aspects and does not necessarily imply that such components must be realized by separate firmware, hardware, software, or the like. Rather, functionality associated with the disclosed components may be performed by separate firmware, hardware, or software elements.

The disclosed techniques may also be embodied or encoded in a computer system-readable medium, such as a computer system-readable storage medium, containing instructions. Instructions embedded or encoded in a computer system-readable medium, including a computer system-readable storage medium, may cause one or more programmable processors, or other processors, to implement one or more of the disclosed techniques, such as when instructions included or encoded in the computer system-readable medium are executed by the one or more processors. Computer system readable storage media may include electronically erasable programmable read only memory (EEPROM), erasable programmable read only memory (EPROM), flash memory, programmable read only memory (PROM), random access memory (RAM), read only memory (ROM), a hard disk, a compact disc ROM (CD-ROM), a floppy disk, a cassette, magnetic media, optical media, or other computer system readable media. In some examples, an article of manufacture may comprise one or more computer system-readable storage media containing instructions to perform one or more of the techniques disclosed herein.

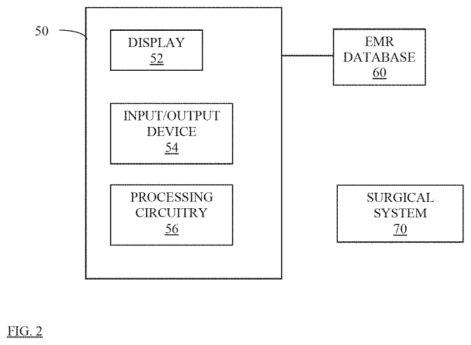

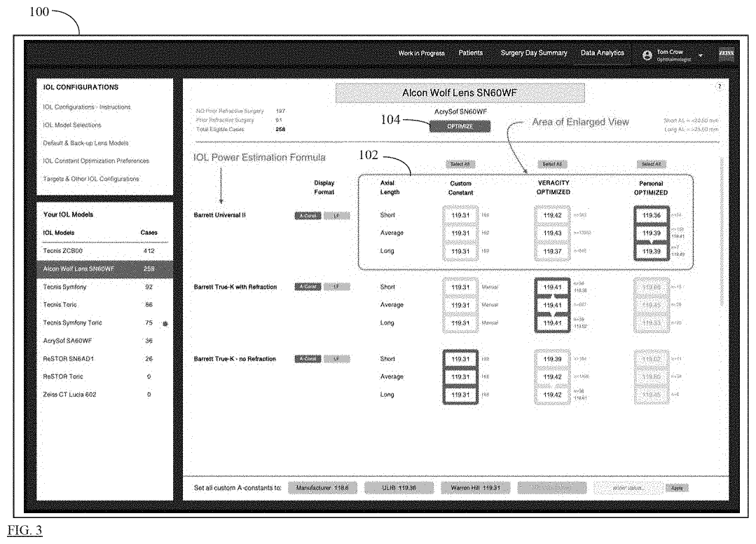

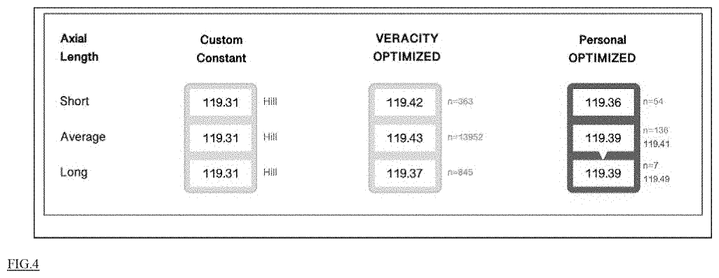

FIG. 3 is a depiction of a graphical user interface 100 (e.g., ZEISS VERACITY Surgical user interface) according to an example embodiment of the invention. User interface 100 includes an example set of constants for one IOL model, AcrySof SN60WF. The user interface 100 depicts IOL constants for three IOL power estimation formulas including, Barrett Universal II, Barrett True-K with refraction, and Barrett True-K with no Refraction. However, other power estimation formulas may also be used. Within each IOL power estimation formula, three IOL constants (e.g., custom/manual inputted constant, VERACITY optimized constant and personal optimized constant) are depicted for three representative axial lengths (e.g., short, average, and long axial eye lengths). FIG. 4 is an enlarged view of area 102 containing a detailed view of the graphical user interface depicted in FIG. 3 for the optimized IOL constants determined for Barrett Universal II IOL power estimation formula.

The constants displayed are based on example results for both personal and globally conducted IOL constant analysis in accordance with the method of FIG. 1. As best seen in FIG. 4, the displayed and previously selected personal IOL constant for the Barrett Universal II formula for an eye of average axial length is selection is 119.39. The 119.39 personal IOL constant represents a previously optimized constant for the specific user which may have been manually selected by the user before additional surgical cases yielded a slightly different constant. As such, the 119.39 represent an out-of-date or unoptimized IOL constant for the user. To the side of the displayed IOL constant are the displayed values 119.41 and n=136. The 119.41 value represents the calculated optimized IOL constant for the specific user based on updated information from surgical cases performed by the user using the optimization techniques disclosed herein. The n=136 signifies the number of eligible surgical cases performed by that surgeon that were taken into account as part of the available data set used to calculate the optimization IOL constant 119.41. Additional optimized IOL constants are displayed for different axial length groups, differ categorization techniques (e.g., personal vs global), and different IOL power estimation formulas.

The user interface 100 includes an “OPTIMIZE” button 104 that the user may select to update the personal IOL constant for the user to the optimized IOL constant based on the most recent data (e.g., 119.41). The updated value may be used by the user to determine a target IOL for a patient that can be implanted by an appropriate surgical procedure.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method of determining a target IOL based on an optimized IOL constant, comprising with a computing device:
sorting available refraction data from a plurality of cataract surgery patients to identify appropriate post-operative refraction results based on user configurable criteria;
for each identified appropriate post-operative refraction result, determining a reference IOL constant that would have given an exact desired result for a selected IOL power estimation formula;
analyzing the reference IOL constants to provide at least one optimized IOL constant that is:
sub-grouped according to an axial length for the selected IOL power estimation formula; or
determined as a function of axial length;
determining based on the at least one optimized IOL constant, a target IOL power for a target axial length and a target IOL power estimation formula; and
providing, based on the target IOL power, a target IOL implant.

2. The method as claimed in claim 1, further comprising gathering the post-operative refraction data from pre-existing electronic medical records by computer-based analysis via the computing device.

3. The method as claimed in claim 1, further comprising performing separate computerized analysis and optimization for short, long and average axial length eyes.

4. The method as claimed in claim 1, wherein analyzing the reference IOL constants to provide at least one optimized IOL constant is sub-grouped according to an axial length for the selected IOL power estimation formula.

5. The method as claimed in claim 4, wherein analyzing the reference IOL constants to provide at least one optimized IOL constant further comprises, for each sub-group:
determining a mean IOL constant for the sub-group using the reference IOL constants;
excluding a set of outlier reference IOL constants that are at least two or more standard deviations from the mean IOL constant;
recalculating a second mean IOL constant from the remaining values that have not been excluded; and
reporting the recalculated mean IOL constant as the optimized IOL constant.

6. The method as claimed in claim 5, further comprising outputting separate optimized IOL constants for at least short, long and average axial length sub-groups.

7. The method as claimed in claim 1, wherein analyzing the reference IOL constants to provide at least one optimized IOL constant is determined as a function of axial length.

8. The method as claimed in claim 7, wherein analyzing the reference IOL constants to provide at least one optimized IOL constant comprises preforming linear regression analysis on the reference IOL constants to provide an optimized IOL constant determined as a function of axial length:

9. The method as claimed in claim 8, further comprising:
receiving a user selected target axial length; and
determining an optimized IOL constant using the user selected target axial length.

10. The method of claim 1, wherein the target IOL power estimation formula is selected from a group consisting of Barrett Universal II, Barrett True-K with refraction, Barrett True-K with no Refraction, Holladay I, Hoffer II, SRK/T, Haigis, Holladay II and Olson.

11. The method of claim 1, further comprising ordering the target IOL.

12. A non-transitory computer readable data storage medium that is not a carrier wave or signal comprising program code to carry out the method of claim 1.

13. A computerized planning device configured to determine a target IOL based on an optimized IOL constant, comprising a display unit; a user input device; and processing circuitry, the processing circuitry configured to:
sort available refraction data from an EMR database comprising available refraction data from a plurality of cataract surgery patients to identify appropriate post-operative refraction results based on user configurable criteria;
determine a reference IOL constant for each identified appropriate post-operative refraction result that would have given an exact desired result for a selected IOL power estimation formula;
calculate, based on the reference IOL constants, at least one optimized IOL constant that is:
sub-grouped according to an axial length for the selected IOL power estimation formula; or
determined as a function of axial length, and
display the optimized IOL constant.

14. The computerized planning device of claim 12, wherein the processing circuitry is further configured to:
determine based on the at least one optimized IOL constant, a target IOL power for a target axial length and target IOL power estimation formula; and
order based on the target IOL power a target IOL.

15. The computerized planning device of claim 12, wherein the processing circuity is configured to perform separate optimization for short, long and average axial length eyes, and provide at least three optimized IOL constants for each of the short, long and average axial lengths.

16. A computerized method of formula and axial length specific IOL constant optimization, comprising:
electronically sorting available refraction data from a plurality of cataract surgery patients to identify appropriate post operative refraction data based on user configurable criteria;
identifying eligible surgical cases for optimization;
identifying a constant value for each surgical case that would have given an exact desired result with a selected IOL formula;
analyzing a series of the constant values to generate a mean constant value and to define the constant values that deviate by more than two standard deviations from the mean constant value;
excluding the constant values that deviate by more than two standard deviations from the mean;
recalculating a second mean constant value from remaining cases that have not been excluded; and
reporting the recalculated mean constant value as an optimized constant; and
using the optimized constant in at least one future IOL calculation;
using the result of the at least one future IOL calculation to select an IOL for implantation; and
implanting the IOL.

17. The method as claimed in claim 16, further comprising gathering the post operative refraction data from pre-existing electronic medical records by computer-based analysis via a computer interface.

18. The method as claimed in claim 16, further comprising performing separate computerized analysis and optimization for short, long and average axial length eyes.

19. The method as claimed in claim 16, further comprising performing separate computerized analysis and optimization for at least two different IOL calculation formulas.

20. The method as claimed in claim 16, further comprising outputting separate constants for at least short, long and average axial length eyes.

21. The method as claimed in claim 16, further comprising performing separate computerized analysis and optimization to a continuous range of axial lengths through a non-linear regression analysis.
